(19) United States
(12) Patent Application Publication
(10) Pub. No.: US 2008/0199464 A1
(43) Pub. Date: Aug. 21, 2008

(54) Combination Therapy with Angiogenesis Inhibitors

(75) Inventors: Greg Plowman, San Carlos, CA (US); Robert D. Mass, Mill Valley, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/022,318

(22) Filed: Jan. 30, 2008

Related U.S. Application Data
(60) Provisional application No. 60/887,688, filed on Feb. 1, 2007.

(51) Int. Cl. A61K 39/395; A61K 31/711; A61P 35/00

(52) U.S. Cl. 424/133.1; 514/44; 424/141.1

(57) ABSTRACT

Disclosed herein are methods of treating tumors using a combination therapy with angiogenesis inhibitors.

### CROSS-REFERENCE TO RELATED APPLICATION

This application claims the benefit of U.S. Provisional Patent Application No. 60/887,688, filed 1 Feb. 2007, the disclosure of which is incorporated herein by reference in its entirety.

### BACKGROUND OF THE INVENTION

Development of a vascular system is a fundamental requirement for many physiological and pathological processes. Actively growing tissues such as embryos and tumors require adequate blood supply. They satisfy this need by producing pro-angiogenic factors, which promote new blood vessel formation via a process called angiogenesis. Vascular tube formation is a complex but orderly biological event involving all or many of the following steps: a) Endothelial cells (ECs) proliferate from existing ECs or differentiate from progenitor cells; b) ECs migrate and coalesce to form cord-like structures; c) vascular cords then undergo tubulogenesis to form vessels with a central lumen d) existing cords or vessels send out sprouts to form secondary vessels; e) primitive vascular plexus undergo further remodeling and reshaping; and f) peri-endothelial cells are recruited to encase the endothelial tubes, providing maintenance and modulatory functions to the vessels; such cells including pericytes for small capillaries, smooth muscle cells for larger vessels, and myocardial cells in the heart. Hanahan, D. Science 277:48-50 (1997); Hogan, B. L. & Kolodziej, P. A. Nature Reviews Genetics. 3:513-23 (2002); Lubarsky, B. & Krasnow, M. A. Cell. 112:19-28 (2003).

It is now well established that angiogenesis is implicated in the pathogenesis of a variety of disorders. These include solid tumors and metastasis, atherosclerosis, retrolental fibroplasia, hemangiomas, chronic inflammation, intraocular neovascular diseases such as proliferative retinopathies, e.g., diabetic retinopathy, age-related macular degeneration (AMD), neovascular glaucoma, immune rejection of transplanted corneal tissue and other tissues, rheumatoid arthritis, and psoriasis. Folkman et al., J. Biol. Chem., 267:10931-10934 (1992); Klagsbrun et al., Annu. Rev. Physiol. 53:217-239 (1991); and Garner A., “Vascular diseases”, In: Pathobiology of Ocular Disease. A Dynamic Approach, Garner A., Klintworth G K, eds., 2nd Edition (Marcel Dekker, NY, 1994), pp 1625-1710.

In the case of tumor growth, angiogenesis appears to be crucial for the transition from hyperplasia to neoplasia, and for providing nourishment for the growth and metastasis of the tumor. Folkman et al., Nature 339:58 (1989). Neovascularization allows the tumor cells to acquire a growth advantage and proliferative autonomy compared to the normal cells. A tumor usually begins as a single aberrant cell which can proliferate only to a size of a few cubic millimeters due to the distance from available capillary beds, and it can stay ‘dormant’ without further growth and dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. Accordingly, a correlation has been observed between density of microvessels in tumor sections and patient survival in breast cancer as well as in several other tumors. Weidner et al., N. Engl. J. Med 324:1-6 (1991); Horak et al., Lancet 340:1120-1124 (1992); Macchiarini et al., Lancet 340:145-146 (1992). The precise mechanisms that control the angiogenic switch is not well understood, but it is believed that neovascularization of tumor mass results from the net balance of a multitude of angiogenesis stimulators and inhibitors (Folkman, 1995, Nat Med 1(1):27-31).

The process of vascular development is tightly regulated. To date, a significant number of molecules, mostly secreted factors produced by surrounding cells, have been shown to regulate EC differentiation, proliferation, migration and coalescence into cord-like structures. For example, vascular endothelial growth factor (VEGF) has been identified as the key factor involved in stimulating angiogenesis and in inducing vascular permeability. Ferrara et al., Endocr. Rev. 18:4-25 (1997). The finding that the loss of even a single VEGF allele results in embryonic lethality points to an irreplaceable role played by this factor in the development and differentiation of the vascular system. Furthermore, VEGF has been shown to be a key mediator of neovascularization associated with tumors and intraocular disorders. Ferrara et al., Endocr. Rev. supra. The VEGF mRNA is overexpressed by the majority of human tumors examined. Berkman et al., J. Clin. Invest. 91:153-159 (1993); Brown et al., Human Pathol. 26:86-91 (1995); Brown et al., Cancer Res. 53:4727-4735 (1993); Mattern et al., Brit. J. Cancer 73:931-934 (1996); Dvorak et al., Am. J. Pathol. 146:1029-1039 (1995).

Anti-VEGF neutralizing antibodies suppress the growth of a variety of human tumor cell lines in nude mice (Kim et al., Nature 362:841-844 (1993); Warren et al., J. Clin. Invest. 95:1789-1797 (1995); Borgström et al., Cancer Res. 56:4032-4039 (1996); Melnyk et al., Cancer Res. 56:921-924 (1996)) and also inhibit intraocular angiogenesis in models of ischemic retinal disorders. Adamis et al., Arch. Opthalmol. 114:66-71 (1996). Therefore, anti-VEGF monoclonal antibodies or other inhibitors of VEGF action are promising candidates for the treatment of tumors and various intraocular neovascular disorders. Such antibodies are described, for example, in EP 817,648 published Jan. 14, 1998; and in WO98/45331 and WO98/45332, both published Oct. 15, 1998.

Despite the significant advancement in the treatment of cancer achieved by angiogenesis inhibitors such as anti-VEGF antibody, improved therapies are still being sought, especially those that further enhance the overall efficacy.

### SUMMARY OF THE INVENTION

The present invention provides combination therapies for treating tumors, wherein a VEGF antagonist is combined with a protein kinase inhibitor having at least the PDGFR blocking activity, thereby producing anti-tumor activities. In certain embodiments, the VEGF antagonist is a compound that interferes with the binding of VEGF to a cellular receptor. Examples of such VEGF blocking antagonists include, but are not limited to, soluble VEGF receptors, apatmers or peptibodies that are specific to VEGF, and anti-VEGF antibodies. In one embodiment, the anti-VEGF antibody is bevacizumab.

In one aspect, the protein kinase inhibitor is specific to PDGFR. In other aspects, the protein kinase inhibitor targets multiple RTKs including PDGFR and VEGFR-2, thereby blocking both PDGF and VEGF pathways. In one embodiment, the protein kinase inhibitor is sunitinib (SUTENT®).

Methods of the invention can be used for treating different cancers, both solid tumors and soft-tissue tumors alike. Non-limiting examples of cancers amendable to the treatment of the invention include breast cancer, colorectal cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In certain aspects, the cancers are metastatic. In other aspects, the cancers are non-metastatic.

In certain embodiments, bevacizumab and sunitinib are used in combination therapies of cancers such as renal cell carcinoma, non-small cell lung carcinoma, colorectal carcinoma, breast carcinoma or pancreatic carcinoma. In certain embodiments, when used in combination, bevacizumab is administered in the range from about 0.05 mg/kg to about 15 mg/kg. In one embodiment, one or more doses of about 0.5 mg/kg, 1.0 mg/kg, 2.0 mg/kg, 3.0 mg/kg, 4.0 mg/kg, 5.0 mg/kg, 6.0 mg/kg, 7.0 mg/kg, 7.5 mg/kg, 8.0 mg/kg, 9.0 mg/kg, 10 mg/kg or 15 mg/kg (or any combination thereof) may be administered to the subject. Such doses may be administered intermittently, e.g. every day, every three days, every week or every two to three weeks. In another embodiment, when used in combination, bevacizumab is administered intravenously to the subject at 10 mg/kg every other week or 15 mg/kg every three weeks, and sunitinib is administered orally to the subject at a daily dose of about 25 mg to about 50 mg for 1 to 4 weeks on followed by 1 to 2 weeks off. In yet another embodiment, sunitinib is administered at 25 mg/day for 2 weeks followed by 1 week off.

Depending on the specific cancer indication to be treated, the combination therapy of the invention can be combined with additional therapeutic agents, such as chemotherapeutic agents, or additional therapies such as radiotherapy or surgery. Many known chemotherapeutic agents can be used in the combination therapy of the invention. In certain embodiments, the combination therapy of the invention can be combined with more than one chemotherapeutic agent. In one embodiment, the combination therapy of the invention is combined with chemotherapeutic agent paclitaxel. In another embodiment, the combination therapy of the invention is combined with chemotherapeutic agents carboplatin and paclitaxel. In certain embodiments, those chemotherapeutic agents that are standard for the treatment of the specific indications will be used. In another embodiment, dosage or frequency of each therapeutic agent to be used in the combination is the same as, or less than, the dosage or frequency of the corresponding agent when used without the other agent(s).

### BRIEF DESCRIPTION OF THE DRAWINGS

FIGS. 1A and 1B depict growth inhibition of established LS174T human colon carcinoma xenografts in athymic nude mice (n=10 for each group). Administration schedules for sunitinib and anti-VEGF (the MAb B20-4.1) are indicated by arrowheads and arrows, respectively. See Example 1 for dosing details. FIG. 1B is a Kaplan-Meier plot indicating survival.

FIGS. 2A and 2B depict growth inhibition of established H1299 human non-small cell lung carcinoma (NSCLC) xenografts in athymic nude mice (n=10 for each group). FIG. 2B is a Kaplan-Meier plot indicating survival.

FIG. 3 shows the result of another H1299 xenograft growth inhibition study (n=10 for each group). Two different sunitinib doses were used for both monotherapy and combination. See Example 1 for dosing details.

FIG. 4 depicts growth inhibition of established 786-O renal cell carcinoma (RCC) xenografts in athymic nude mice (n=10 for each group).

FIGS. 5A and 5B depict growth inhibition of established Bx-PC3 human pancreatic carcinoma xenografts in athymic nude mice (n=10 for each group). Two different sunitinib doses were used for both monotherapy and combination. See Example 1 for dosing details. FIG. 5B is a Kaplan-Meier plot indicating survival.

FIG. 6 depicts growth inhibition of established Caki-2 renal cell carcinoma (RCC) xenografts in SCID mice (n=10 for each group). Two different sunitinib doses were used for both monotherapy and combination. See Example 1 for dosing details.

FIGS. 7A and 7B summarize and compare the growth inhibition of the Caki-2 RCC xenografts (7A) and the H1299 NSCLC xenografts (7B). In each figure, only the low sunitinib dose results are shown. See Example 1 for dosing details.

FIGS. 8A-C illustrate tumor's morphological changes in treated H1299 NSCLC xenografts. 8A shows the degrees of tumor necrosis under different treatments. Percentages of necrosis are measured by H&E staining; 8B represents the changes of vascular density under different treatments, as measured by PECAM IHC; and 8C shows the tumor and tumor vasculature in a H1299 xenograft treated with the anti-VEGF (B20-4.1) and sunitinib combination.

### DETAILED DESCRIPTION

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The term “VEGF” or “VEGF-A” is used to refer to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof. VEGF-A is part of a gene family including VEGF-B, VEGF-C, VEGF-D, VEGF-E, VEGF-F, and PlGF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. Additionally, neuropilin-1 has been identified as a receptor for heparin-binding VEGF-A isoforms, and may play a role in vascular development. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

A “native sequence” polypeptide comprises a polypeptide having the same amino acid sequence as a polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

An “angiogenesis inhibitor” or “anti-angiogenesis agent” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or to the VEGF-A receptor (e.g., KDR receptor or Flt-1 receptor), anti-PDGFR inhibitors such as GLEEVEC® (Imatinib Mesylate). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials.

A “VEGF antagonist” refers to a molecule (peptidyl or non-peptidyl) capable of neutralizing, blocking, inhibiting, abrogating, reducing, or interfering with VEGF activities including its binding to one or more VEGF receptors. In certain embodiments, the VEGF antagonist reduces or inhibits, by at least 10%, 20%, 30%, 40%, 50%, 60%, 70%, 80%, 90% or more, the expression level or biological activity of VEGF. In one embodiment, the VEGF inhibited by the VEGF antagonist is VEGF (8-109), VEGF (1-109), or VEGF₁₆₅. VEGF antagonists useful in the methods of the invention include peptidyl or non-peptidyl compounds that specifically bind VEGF, such as anti-VEGF antibodies and antigen-binding fragments thereof, polypeptides, or fragments thereof that specifically bind to VEGF, and receptor molecules and derivatives that bind specifically to VEGF thereby sequestering its binding to one or more receptors (e.g., soluble VEGF receptor proteins, or VEGF binding fragments thereof, or chimeric VEGF receptor proteins); antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers.

An “anti-VEGF antibody” is an antibody that binds to VEGF with sufficient affinity and specificity. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-VEGF antibody of the invention can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay (as described in the Examples below); tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PlGF, PDGF or bFGF.

In certain embodiments, anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. Cancer Res. 57:4593-4599 (1997). In one embodiment, the anti-VEGF antibody is “Bevacizumab (BV)”, also known as “rhuMAb VEGF” or “AVASTIN®” It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab has been approved by the FDA for use in combination with chemotherapy regimens to treat metastatic colorectal cancer (CRC) and non-small cell lung cancer (NSCLC). Hurwitz et al., N. Engl. J. Med. 350:2335-42 (2004); Sandler et al., N. Engl. J. Med. 355:2542-50 (2006). Currently, bevacizumab is being investigated in many ongoing clinical trials for treating various cancer indications. Kerbel, J. Clin. Oncol. 19:45 S-51S (2001); De Vore et al, Proc. Am. Soc. Clin. Oncol. 19:485a. (2000); Hurwitz et al., Clin. Colorectal Cancer 6:66-69 (2006); Johnson et al., Proc. Am. Soc. Clin. Oncol. 20:315a (2001); Kabbinavar et al. J. Clin. Oncol. 21:60-65 (2003); Miller et al., Breast Can. Res. Treat. 94:Suppl 1:S6 (2005).

Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, and U.S. Patent Application 60/991,302, the content of these patent applications are expressly incorporated herein by reference. For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, I83 and Q89.

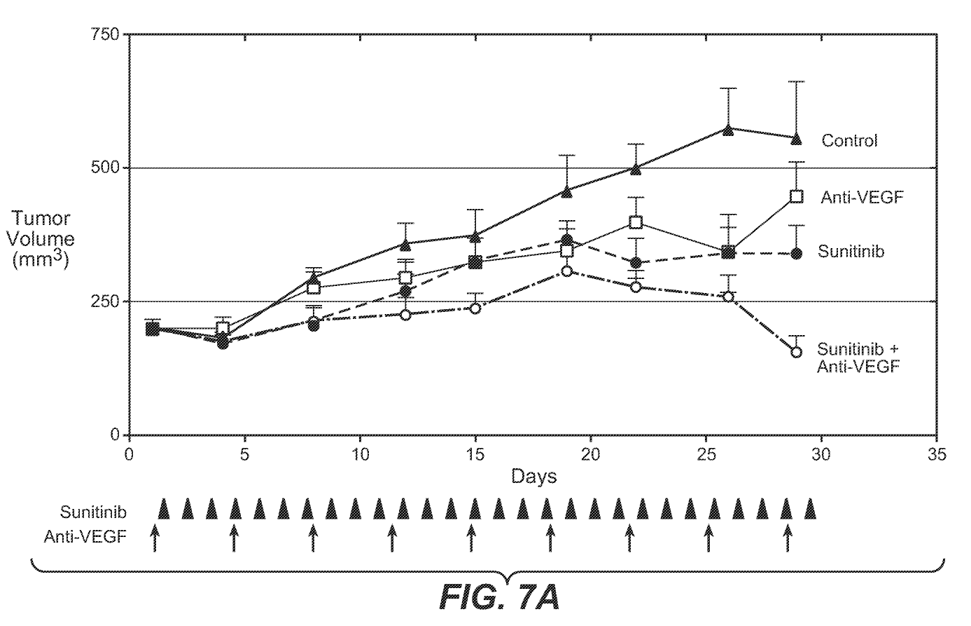

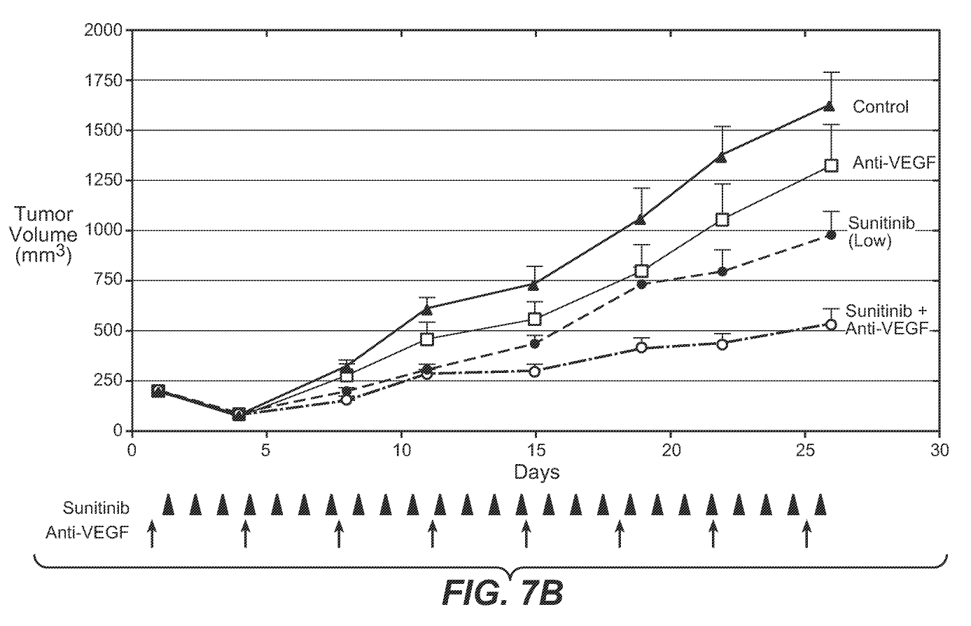

A “G6 series antibody” according to this invention, is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, I83 and Q89.

A “B20 series antibody” according to this invention is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and U.S. Patent Application 60/991,302, the content of these patent applications are expressly incorporated herein by reference. In one embodiment, the B20 series antibody binds to a functional epitope on human VEGF comprising residues F17, M18, D19, Y21, Y25, Q89, I91, K101, E103, and C104.

A “functional epitope” according to this invention refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC50 mutant VEGF/IC50 wild-type VEGF) of the antibody will be greater than 5 (see Example 2 of WO2005/012359). In one embodiment, the relative affinity ratio is determined by a solution binding phage displaying ELISA. Briefly, 96-well Maxisorp immunoplates (NUNC) are coated overnight at 4° C. with an Fab form of the antibody to be tested at a concentration of 2 ug/ml in PBS, and blocked with PBS, 0.5% BSA, and 0.05% Tween20 (PBT) for 2 h at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′,5,5′-tetramethylbenzidine (TMB, Kirkegaard & Perry Labs, Gaithersburg, Md.) substrate for approximately 5 min, quenched with 1.0 M H3PO4, and read spectrophotometrically at 450 nm. The ratio of IC50 values (IC50, ala/IC50, wt) represents the fold of reduction in binding affinity (the relative binding affinity).

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The “Kd” or “Kd value” according to this invention is in one embodiment measured by a radiolabeled VEGF binding assay (RIA) performed with the Fab version of the antibody and a VEGF molecule as described by the following assay that measures solution binding affinity of Fabs for VEGF by equilibrating Fab with a minimal concentration of (¹²⁵I)-labeled VEGF(109) in the presence of a titration series of unlabeled VEGF, then capturing bound VEGF with an anti-Fab antibody-coated plate (Chen, et al., (1999) J. Mol. Biol 293:865-881). To establish conditions for the assay, microtiter plates (Dynex) are coated overnight with 5 ug/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [¹²⁵I]VEGF(109) are mixed with serial dilutions of a Fab of interest, e.g., Fab-12 (Presta et al., (1997) Cancer Res. 57:4593-4599). The Fab of interest is then incubated overnight; however, the incubation may continue for 65 hours to insure that equilibrium is reached. Thereafter, the mixtures are transferred to the capture plate for incubation at room temperature for one hour. The solution is then removed and the plate washed eight times with 0.1% Tween-20 in PBS. When the plates had dried, 150 ul/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. According to another embodiment the Kd or Kd value is measured by using surface plasmon resonance assays using a BIAcore™-2000 or a BIAcore™-3000 (BIAcore, Inc., Piscataway, N.J.) at 25° C. with immobilized hVEGF (8-109) CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Human VEGF is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of human VEGF, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) was calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate is can be determined by using a fluorescent quenching technique that measures the increase or decrease in fluorescence emission intensity (excitation=295 nm; emission=340 nm, 16 nm band-pass) at 25° C. of a 20 nM anti-VEGF antibody (Fab form) in PBS, pH 7.2, in the presence of increasing concentrations of human VEGF short form (8-109) or mouse VEGF as measured in a spectrometer, such as a stop-flow equipped spectrophometer (Aviv Instruments) or a 8000-series SLM-Aminco spectrophotometer (ThermoSpectronic) with a stirred cuvette.

A “blocking” antibody or an antibody “antagonist” is one which inhibits or reduces biological activity of the antigen it binds. For example, a VEGF-specific antagonist antibody binds VEGF and inhibits the ability of VEGF to induce vascular endothelial cell proliferation. In certain embodiments, blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10): 1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K_{d}) value of no more than about 1×10⁻⁷ M, preferably no more than about 1×10⁻⁸ M and most preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above. In one embodiment, the species-dependent antibody is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

To increase the half-life of the antibodies or polypeptide containing the amino acid sequences of this invention, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (e.g., Ghetie et al., Ann. Rev. Immunol. 18:739-766 (2000), Table 1). Antibodies with substitutions in an Fc region thereof and increased serum half-lives are also described in WO00/42072, WO 02/060919; Shields et al., J. Biol. Chem. 276:6591-6604 (2001); Hinton, J. Biol. Chem. 279:6213-6216 (2004)). In another embodiment, the serum half-life can also be increased, for example, by attaching other polypeptide sequences. For example, antibodies or other polypeptides useful in the methods of the invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one preferred embodiment, the serum albumin peptide to be attached comprises an amino acid sequence of DICLPRWGCLW. In another embodiment, the half-life of a Fab is increased by these methods. See also, Dennis et al. J. Biol. Chem. 277:35035-35043 (2002) for serum albumin binding peptide sequences.

A “chimeric VEGF receptor protein” is a VEGF receptor molecule having amino acid sequences derived from at least two different proteins, at least one of which is as VEGF receptor protein. In certain embodiments, the chimeric VEGF receptor protein is capable of binding to and inhibiting the biological activity of VEGF.

An “isolated” polypeptide or “isolated” antibody is one that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or nonproteinaceous solutes. In one embodiments, the polypeptide or antibody will be purified (1) to greater than 95% by weight of polypeptide or antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide or antibody includes the polypeptide or antibody in situ within recombinant cells since at least one component of the polypeptide's natural environment will not be present. Ordinarily, however, isolated polypeptide or antibody will be prepared by at least one purification step.

By “fragment” is meant a portion of a polypeptide or nucleic acid molecule that contains, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more nucleotides or 10, 20, 30, 40, 50, 60, 70, 80, 90, 100, 120, 140, 160, 180, 190, 200 amino acids or more.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer.

The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation.

By “dysplasia” is meant any abnormal growth or development of tissue, organ, or cells. Preferably, the dysplasia is high grade or precancerous.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass.

Both stimulatory and inhibitory molecular pathways within the tumor cell regulate this behavior, and interactions between the tumor cell and host cells in the distant site are also significant.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

By “primary tumor” or “primary cancer” is meant the original cancer and not a metastatic lesion located in another tissue, organ, or location in the subject's body.

By “benign tumor” or “benign cancer” is meant a tumor that remains localized at the site of origin and does not have the capacity to infiltrate, invade, or metastasize to a distant site.

By “tumor burden” is meant the number of cancer cells, the size of a tumor, or the amount of cancer in the body. Tumor burden is also referred to as tumor load.

By “tumor number” is meant the number of tumors.

By “subject” is meant a mammal, including, but not limited to, a human or non-human mammal, such as a bovine, equine, canine, ovine, or feline. In one embodiment, the subject is a human.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl, 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above.

“Protein kinases” refers to a large class of enzymes which catalyze the transfer of the γ-phosphate from ATE to the hydroxyl group on the side chain of Ser/Thr or Tyr in proteins and peptides and are intimately involved in the control of various important cell functions, most notably signal transduction, differentiation, and proliferation. Although each of these protein kinases phosphorylate particular protein/peptide substrates, they all bind the same second substrate ATP in a highly conserved pocket. A number of diseases, notably including cancer, are linked to perturbation of protein kinase-mediated cell signaling pathway.

“Protein kinase inhibitors” refers to large or small molecular weight compounds capable of blocking one or more protein kinase activities. In certain embodiments, protein kinase inhibitors are small molecule tyrosine kinase inhibitors (TKIs) that target one or more receptor tyrosine kinases that are implicated in tumor growth, pathologic angiogenesis and metastatic progression of cancer. TKIs target the intracellular kinase domain of the receptor, thereby reducing or shutting down the signal transduction. In addition to inhibiting VEGFR, many of the currently developed small molecule TKIs target other receptors, especially those in the split kinase domain family of receptor tyrosine kinases. Examples of receptor tyrosine kinases include epidermal growth factor receptor (EGFR), vascular endothelial growth factor receptor (VEGFR), platelet-derived growth factor receptor (PDGFR) and fibroblast growth factor receptor (FGFR). Platelet-derived growth factor (PDGF) is another key mediator of tumor-related angiogenesis. It is secreted by many tumors in a paracrine fashion, and is believed to promote endothelial cell proliferation and stroma formation. Similar to VEGF, the production of PDGF is up regulated under low oxygen conditions such as those found in poorly vascularized tumor tissue. PDGF promote tumor growth via multiple processes, including autocrine stimulation of cancer cells and paracrine stimulation of stromal cells. Heldin et al., Physiol Rev 79-1283-1316 (1999); Sundberg et al., Am J Pathol 151:479-492 (1997). Many therapeutic small molecule TKIs are known in the art, including, but are not limited to, vatalanib (PTK787), erlotinib (TARCEVA®), OSI-7904, ZD6474 (ZACTIMA®), ZD6126 (ANG453), ZD1839, sunitinib (SUTENT®), semaxanib (SU5416), AMG706, AG013736, Imatinib (GLEEVEC®), MLN-518, CEP-701, PKC-412, Lapatinib (GSK572016), VELCADE®, AZD2171, sorafenib (NEXAVAR®), XL880, and CHIR-265.

The term “pharmaceutically acceptable salt form” refers to those salt forms that retain the biological effectiveness and properties of the active compound such as sunitinib. Such salts include: (1) acid addition salt which is obtained by reaction of the free base of the parent compound with inorganic acids such as hydrochloric acid, hydrobromic acid, nitric acid, 5 phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of sunitinib; or (2) salts formed when an acidic proton present in the parent compound either is replaced by a metal ion, e.g., an alkali metal ion, an alkaline earth ion; or coordinates with an organic base. Exemplary ions include aluminum, calcium, lithium, magnesium, potassium, sodium and zinc in their usual valences. Preferred organic base include protonated tertiary 15 amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, the size of the primary tumor, or the size or number of the blood vessels in angiogenic disorders.

### Therapeutic Agents

The present invention features the use of VEGF antagonists and protein kinase inhibitors in combination therapy to treat tumor in a subject. A VEGF antagonist refers to a molecule capable of binding to VEGF, reducing VEGF expression levels, or neutralizing, blocking, inhibiting, abrogating, reducing, or interfering with VEGF biological activities, including VEGF binding to one or more VEGF receptors and VEGF mediated angiogenesis and endothelial cell survival or proliferation. Included as VEGF-antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF antagonists also include antagonistic variants of VEGF polypeptides, RNA aptamers and peptibodies against VEGF. Examples of each of these are described below.

Anti-VEGF antibodies that are useful in the methods of the invention include any antibody, or antigen binding fragment thereof, that bind with sufficient affinity and specificity to VEGF and can reduce or inhibit the biological activity of VEGF. An anti-VEGF antibody will usually not bind to other VEGF homologues such as VEGF-B or VEGF-C, nor other growth factors such as PlGF, PDGF, or bFGF. Examples of such anti-VEGF antibodies include, but not limited to, those provided herein under “Definitions.”

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules or fragments thereof that specifically bind to VEGF can be used in the methods of the invention to bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the flt-1 or KDR receptor), that is capable of binding to and inhibiting the biological activity of VEGF. In certain embodiments, the chimeric VEGF receptor proteins of the present invention consist of amino acid sequences derived from only two different VEGF receptor molecules; however, amino acid sequences comprising one, two, three, four, five, six, or all seven Ig-like domains from the extracellular ligand-binding region of the flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include soluble Flt-1/Fc, KDR/Fc, or FLt-1/KDR/Fc (also known as VEGF Trap). (See for example PCT Application Publication No. WO97/44453).

A soluble VEGF receptor protein or chimeric VEGF receptor proteins of the present invention includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

Aptamers are nucleic acid molecules that form tertiary structures that specifically bind to a target molecule, such as a VEGF polypeptide. The generation and therapeutic use of aptamers are well established in the art. See, e.g., U.S. Pat. No. 5,475,096. A VEGF aptamer is a pegylated modified oligonucleotide, which adopts a three-dimensional conformation that enables it to bind to extracellular VEGF. One example of a therapeutically effective aptamer that targets VEGF for treating age-related macular degeneration is pegaptanib (Macugen™, OSI). Additional information on aptamers can be found in U.S. Patent Application Publication No. 20060148748.

A peptibody is a peptide sequence linked to an amino acid sequence encoding a fragment or portion of an immunoglobulin molecule. Polypeptides may be derived from randomized sequences selected by any method for specific binding, including but not limited to, phage display technology. In one embodiment, the selected polypeptide may be linked to an amino acid sequence encoding the Fc portion of an immunoglobulin. Peptibodies that specifically bind to and antagonize VEGF are also useful in the methods of the invention.

The protein kinase inhibitors useful in the present invention are those having at least the ability to block the PDGF signaling pathway, by targeting a PDGFR tyrosine kinase. In certain embodiments, the inhibitors are small molecule, non-peptide compounds. In one embodiment, the protein kinase inhibitors of the invention target both PDGFR and VEGFR-2 tyrosine kinases. An example of the PDGFR/VEGFR-2 dual inhibitor is sunitinib.

Sunitinib (SUTENT®, SU11248, Pfizer Inc) is an oral inhibitor targeting several related protein tyrosine kinase receptors, including PDGFR-beta, KIT, and FLT-3, as well as the three VEGF receptors. SUTENT® is the malate salt of sunitinib. Sunitinib malate is described chemically as butanedioic acid, hydroxy-(2S)—, compounded with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamde (1:1). The molecule formula is C₂₂H₂₇FN₄O₂—C₄H₆O₅. Sun et al., J Med Chem 41:2588-2603 (1998). Preclinical studies have shown that sunitinib has antiangiogenic effects mediated through VEGFR and PDGFR-beta and direct antitumor activity through KIT in various tumor cell lines. Abrams et al., Mol Cancer Ther 2:1011-21 (2003); O'Farrell et al., Blood 101:3597-605 (2003). A recent study in Lewis lung carcinoma tumors demonstrated that sunitinib slows the progression of tumor growth and attenuated the development of metastases, although it did not cause regression of primary tumors. Osusky et al., Angiogenesis 7:225-33 (2004). Based on preclinical/clinical evidence, the mechanism of activity for sunitinib in RCC is thought to be through dual inhibition of the VEGF pathway in endothelial cells and PDGFR-beta expressed on the supporting pericytes. Motzer et al., JAMA 295:2516-24 (2006). Sunitinib has recently been approved in the U.S. for use in advanced renal cell carcinoma and gastrointestinal stromal tumors (GIST).

### Combination Therapies

The present invention features the combination use of a VEGF antagonist and a protein kinase inhibitor as part of a specific treatment regimen intended to provide a beneficial effect from the combined activity of these therapeutic agents. The beneficial effect of the combination includes, but is not limited to, pharmacokinetic or pharmacodynamic co-action resulting from the combination of therapeutic agents. The present invention is particularly useful in treating cancers of various types at various stages.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from the original site and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade, and cancer cells are described as being well-differentiated (low grade), moderately-differentiated, poorly-differentiated, or undifferentiated (high grade). Well-differentiated cells are quite normal appearing and resemble the normal cells from which they originated. Undifferentiated cells are cells that have become so abnormal that it is no longer possible to determine the origin of the cells.

Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

To stage cancer, the American Joint Committee on Cancer first places the cancer, particularly solid tumors, in a letter category using the TNM classification system. Cancers are designated the letter T (tumor size), N (palpable nodes), and/or M (metastases). T1, T2, T3, and T4 describe the increasing size of the primary lesion; N0, N1, N2, N3 indicates progressively advancing node involvement; and M0 and M1 reflect the absence or presence of distant metastases.

In the second staging method, also known as the Overall Stage Grouping or Roman Numeral Staging, cancers are divided into stages 0 to IV, incorporating the size of primary lesions as well as the presence of nodal spread and of distant metastases. In this system, cases are grouped into four stages denoted by Roman numerals I through IV, or are classified as “recurrent.” For some cancers, stage 0 is referred to as “in situ” or “Tis,” such as ductal carcinoma in situ or lobular carcinoma in situ for breast cancers. High grade adenomas can also be classified as stage 0. In general, stage I cancers are small localized cancers that are usually curable, while stage IV usually represents inoperable or metastatic cancer. Stage II and III cancers are usually locally advanced and/or exhibit involvement of local lymph nodes. In general, the higher stage numbers indicate more extensive disease, including greater tumor size and/or spread of the cancer to nearby lymph nodes and/or organs adjacent to the primary tumor. These stages are defined precisely, but the definition is different for each kind of cancer and is known to the skilled artisan.

Many cancer registries, such as the NCI's Surveillance, Epidemiology, and End Results Program (SEER), use summary staging. This system is used for all types of cancer. It groups cancer cases into five main categories:

In situ is early cancer that is present only in the layer of cells in which it began.

Localized is cancer that is limited to the organ in which it began, without evidence of spread.

Regional is cancer that has spread beyond the original (primary) site to nearby lymph nodes or organs and tissues.

Distant is cancer that has spread from the primary site to distant organs or distant lymph nodes.

Unknown is used to describe cases for which there is not enough information to indicate a stage.

In addition, it is common for cancer to return months or years after the primary tumor has been removed. Cancer that recurs after all visible tumor has been eradicated, is called recurrent disease. Disease that recurs in the area of the primary tumor is locally recurrent, and disease that recurs as metastases is referred to as a distant recurrence.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

### Chemotherapeutic Agents

The combination therapy of the invention can further comprise one or more chemotherapeutic agent(s). The combined administration includes coadministration or concurrent administration, using separate formulations or a single pharmaceutical formulation, and consecutive administration in either order, wherein there is a time period while both (or all) active agents simultaneously exert their biological activities.

The chemotherapeutic agent, if administered, is usually administered at dosages known therefor, or optionally lowered due to combined action of the drugs or negative side effects attributable to administration of the antimetabolite chemotherapeutic agent. Preparation and dosing schedules for such chemotherapeutic agents may be used according to manufacturers' instructions or as determined empirically by the skilled practitioner. In one embodiment where the chemotherapeutic agent is paclitaxel, it is administered every week (e.g. at about 60-90 mg/m²) or every 3 weeks (for example at about 135-200 mg/m²). Suitable docetaxel dosages include 60 mg/m², 70 mg/m², 75 mg/m², 100 mg/m² (every 3 weeks); or 35 mg/m² or 40 mg/m² (every week).

Various chemotherapeutic agents that can be combined are disclosed above. In certain embodiments, chemotherapeutic agents to be combined are selected from the group consisting of a taxoid (including docetaxel and paclitaxel), vinca (such as vinorelbine or vinblastine), platinum compound (such as carboplatin or cisplatin), aromatase inhibitor (such as letrozole, anastrazole, or exemestane), anti-estrogen (e.g. fulvestrant or tamoxifen), etoposide, thiotepa, cyclophosphamide, methotrexate, liposomal doxorubicin, pegylated liposomal doxorubicin, capecitabine, gemcitabine, COX-2 inhibitor (for instance, celecoxib), or proteosome inhibitor (e.g. PS342). In one embodiment, the combination therapy of the invention is combined with paclitaxel. In another embodiment, the combination therapy of the invention is combined with carboplatin and paclitaxel.

### Formulations, Dosages and Administrations

The therapeutic agents used in the invention will be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, the drug-drug interaction of the agents to be combined, and other factors known to medical practitioners.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, in one embodiment, the formulation contains a pharmaceutically acceptable salt, such sodium chloride, at about physiological concentrations. Optionally, in another embodiment, the formulations of the invention can contain a pharmaceutically acceptable preservative. In certain embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, in yet another embodiment, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The therapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In the case of VEGF antagonists, local administration is particularly desired if extensive side effects or toxicity is associated with VEGF antagonism. An ex vivo strategy can also be used for therapeutic applications. Ex vivo strategies involve transfecting or transducing cells obtained from the subject with a polynucleotide encoding a VEGF antagonist. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

For example, if the VEGF antagonist is an antibody, the antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. In one embodiment, the dosing is given by injections. In another embodiment, the dosing is given by intravenous or subcutaneous injections, depending in part on whether the administration is brief or chronic.

In another example, the VEGF antagonist compound is administered locally, e.g., by direct injections, when the disorder or location of the tumor permits, and the injections can be repeated periodically. The VEGF antagonist can also be delivered systemically to the subject or directly to the tumor cells, e.g., to a tumor or a tumor bed following surgical excision of the tumor, in order to prevent or reduce local recurrence or metastasis.

Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, the VEGF antagonist in the combination may be administered by intravenous injection while the protein kinase inhibitor in the combination may be administered orally. Alternatively, for example, both the therapeutic agents may be administered orally, or both therapeutic agents may be administered by intravenous injection, depending on the specific therapeutic agents. The sequence in which the therapeutic agents are administered also varies depending on the specific agents.

Depending on the type and severity of the disease, about 1 μg/kg to 100 mg/kg (e.g., 0.1-20 mg/kg) of each therapeutic agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above. However, other dosage regimens may be useful. In one example, if the VEGF antagonist is an antibody, the antibody of the invention is administered every two to three weeks, at a dose ranging from about 5 mg/kg to about 15 mg/kg. If the protein kinase inhibitor is an oral small molecule compound, the drug is administered daily at a dose ranging from about 25 mg/day to about 50 mg/day. Moreover, the oral compound of the invention can be administered either under a traditional high-dose intermittent regimen, or using lower and more frequent doses without scheduled breaks (referred to as “metronomic therapy”). When an intermittent regimen is used, for example, the drug can be given daily for two to three weeks followed by a one week break; or daily for four weeks followed by a two week break, depending on the daily dose and particular indication. The progress of the therapy of the invention is easily monitored by conventional techniques and assays.

The following examples are intended merely to illustrate the practice of the present invention and are not provided by way of limitation. The disclosures of all patent and scientific literatures cited herein are expressly incorporated in their entirety by reference.

### EXAMPLES

### Example 1

### Combination of Anti-VEGF and Sunitinib for Inhibiting Human Tumor Growth In Vivo

This experiment evaluates the anti-VEGF monoclonal antibody B20-4.1 as monotherapy and in combination with the small molecule tyrosine kinase inhibitor sunitinib for activity against various human carcinoma xenografts in nude mice.

### Methods and Materials

The following human tumor cells were used in the xenograft study:

LS174T human colon carcinoma

H1299 human non-small cell lung carcinoma

786-O human renal cell carcinoma

Caki-2 human renal cell carcinoma

Bx-PC3 human pancreatic carcinoma

Female athymic nude mice (10-11 weeks old on Day 1 of the study) or C.B-17 SCID mice (15-16 weeks old on Day 1 of the study) were used. Xenografts were initiated either from cultured human carcinoma cells (in the case of the H1299 cells), or from existing human tumors maintained in xenografted mice. Each test mouse received either tumor cells or tumor fragment implanted subcutaneously in the right flank, and the growth of tumors was monitored. After a period of tumor growth, the length of which depending on the specific human tumors tested, the mice were placed into different groups each consisting of ten mice. Volume was calculated using the formula:

Tumor Volume(mm³)=(W2×l)/2

where w=width and l=length in mm of a human tumor. Tumor weight may be estimated with the assumption that 1 mg is equivalent to 1 mm³ of tumor volume.

A control IgG antibody and the test MAb B20-4.1 as well as sunitinib were used in the study. The control and B20-4.1 antibodies were each administered at a single dose level (5 mg/kg i.p. biweekly to end). Sunitinib was administered at two doses levels (25 or 50 mg/kg p.o. once daily to end).

For combination treatments given to a group on the same day, antibody doses were administered thirty minutes prior to sunitinib doses. Each dose of PBS, control IgG, or B20-4.1 was administered in a volume of 0.2 mL per 20 g body weight (10 mL/kg), and each dose of sunitinib or vehicle was administered in a volume of 0.1 mL per 20 g body weight (5 mL/kg). All doses were scaled to the body weights of the animals.

Tumors were measured twice weekly using calipers. Each animal was euthanized when its tumor reached the endpoint size of 2,000 mm³ or at the termination of the study, whichever comes first. Treatment outcome was based on tumor growth inhibition (TGI). TGI evaluates the data from all animals in a group, excluding animals that die due to treatment-related (TR) or non-treatment-related (NTR) causes, and is defined as the difference between final median tumor volumes of the treatment and control groups, expressed as a percentage of the median tumor volume of the control group. An agent that produces at least 60% TGI in this assay is classified as therapeutically active.

Treatment may cause partial regression (PR) or complete regression (CR) of the tumor in an animal. In a PR response, the tumor volume is 50% or less of its Day 1 volume for three consecutive measurements during the course of the study, and equal to or greater than 13.5 mm³ for one or more of these three measurements. In a CR response, the tumor volume is less than 13.5 mm³ for three consecutive measurements during the course of the study. Animals were monitored for regression responses.

Animals were euthanized when their tumors reached a volume of 2000 mm3 or at the termination of the study. Serum, tumor, and kidney samples were collected for further assays including histologic assessements of tumor and tumor vasculature.

Animals were weighed daily for the first five days of the study and then twice weekly. The mice were observed frequently for overt signs of any adverse, treatment-related side effects, and clinical signs of toxicity were recorded when observed. Acceptable toxicity is defined as a group mean body-weight loss of less than 20% during the study and not more than one treatment-related (TR) death among ten treated animals. Any dosing regimen that results in greater toxicity is considered above the maximum tolerated dose (MID). A death is classified as TR if attributable to treatment side effects as evidenced by clinical signs and/or necropsy, or if due to unknown causes during the dosing period or within 10 days of the last dose. A death is classified as an NTR if there is no evidence that death was related to treatment side effects.

Statistical analysis of differences between median tumor burdens in control and treated groups were analyzed using the Mann-Whitney U-test. Two-tailed statistical analyses were conducted at significance level P=0.05. Results were deemed statistically significant at 0.01≦P≦0.05, and highly significant at P<0.01.

### Results

### Inhibition of LS174T Colon Carcinoma Growth

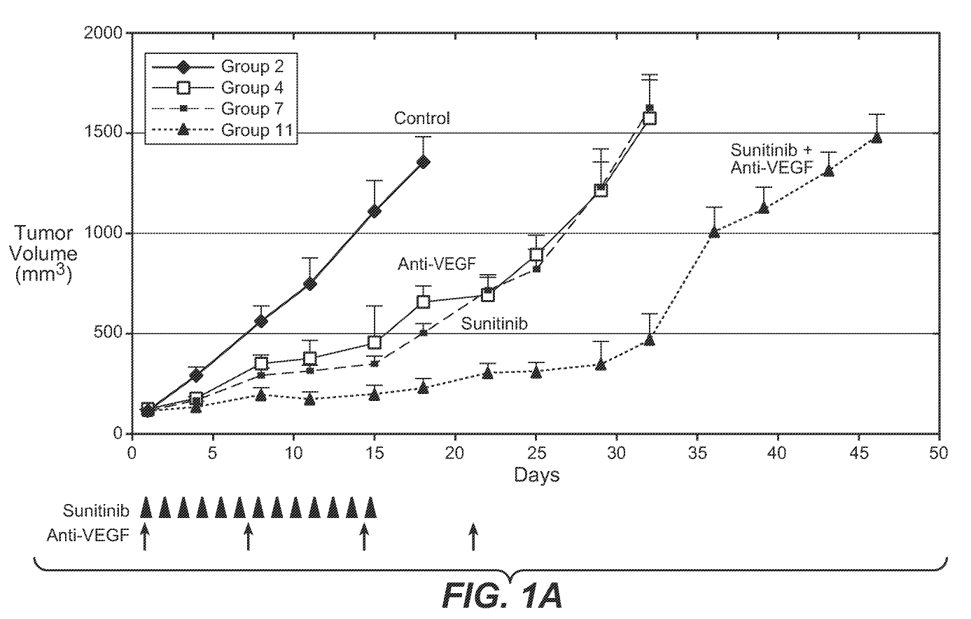

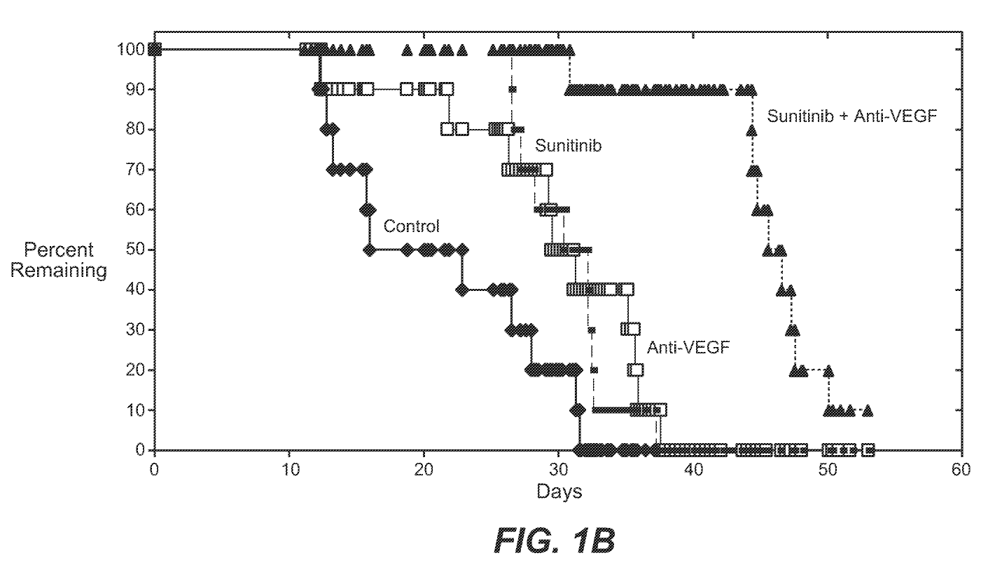

FIG. 1A shows tumor growth in volume over time for each group of mice in the LS174T colon carcinoma study. Group 2 (curve with diamond) was given the control antibody; Group 4 (curve with square) was given 4 doses of B20-4.1 alone; Group 7 (plain curve) was given sunitinib at 50 mg/kg po daily for 14 days; and Group 11 (curve with triangle) was given both B20-4.1 and sunitinib, at the same dosing schedule as the monotherapy groups. FIG. 1B is a Kaplan-Meier plot of the same study results. As shown in both figures, the combination of B20-4.1 and sunitinib produced significantly increased inhibition of the colon tumor growth than either of the single agent treatment.

In a separate 25-day study, a high sunitinib dose (50 mg/kg daily) and a low sunitinib dose (25 mg/kg daily) were used in both monotherapy and combination groups. The MTVs and TGIs of different treatment groups were measured at Day 18. B20-4.1 monotherapy produced a marginal 48% TGI. Sunitinib monotherapy produced dose-dependent responses, with no TGI at low dose and non-significant 43% TGI at high dose. The combination of B20-4.1 with the lower dose sunitinib produced a 50% TGI that was comparable to B20-4.1 monotherapy. The combination of B20-4.1 with the higher dose of sunitinib resulted in a 75% TGI. All treatments appeared to be well-tolerated, with no evidence of toxicity based on body weight measurements or clinical symptoms.

### Inhibition of H1299 NSCLC Growth

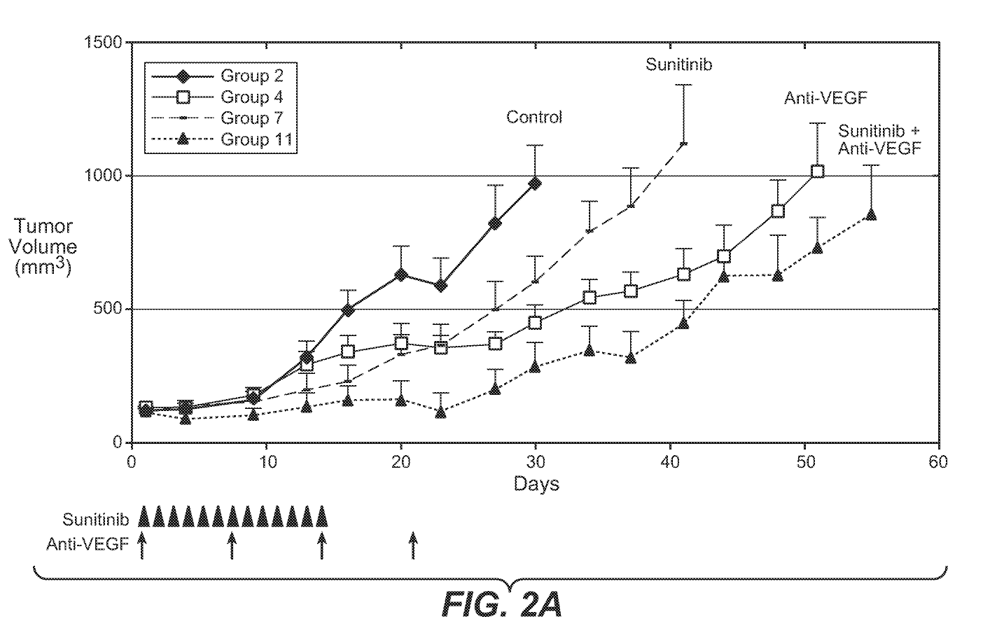

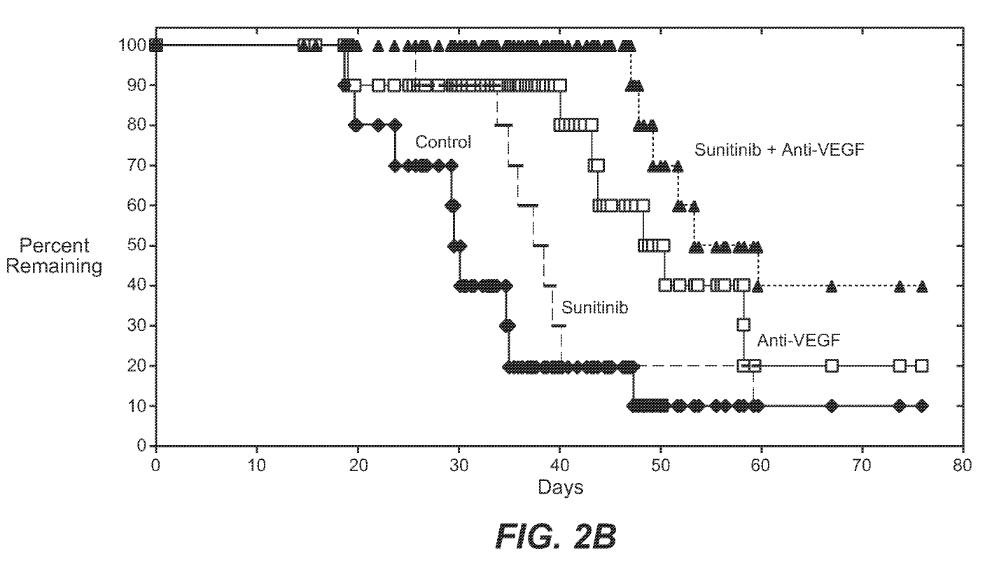

Two studies were conducted for the H1299 non-small cell lung carcinoma (NSCLC) xenografts. FIGS. 2A and 2B represent the results from a short term treatment study that is similar to the LS174T study as described above. Group 2 (curve with diamond) was given the control antibody; Group 4 (curve with square) was given 4 doses of B20-4.1 alone; Group 7 (plain curve) was given sunitinib at 50 mg/kg po daily for 14 days; and Group 11 (curve with triangle) was given both B20-4.1 and sunitinib, at the same dosing schedule as the monotherapy groups. FIG. 2B is a Kaplan-Meier plot of the same study results. As shown in both figures, the combination of B20-4.1 and sunitinib produced significantly increased inhibition of the NSCLC tumor growth than either of the single agent treatment.

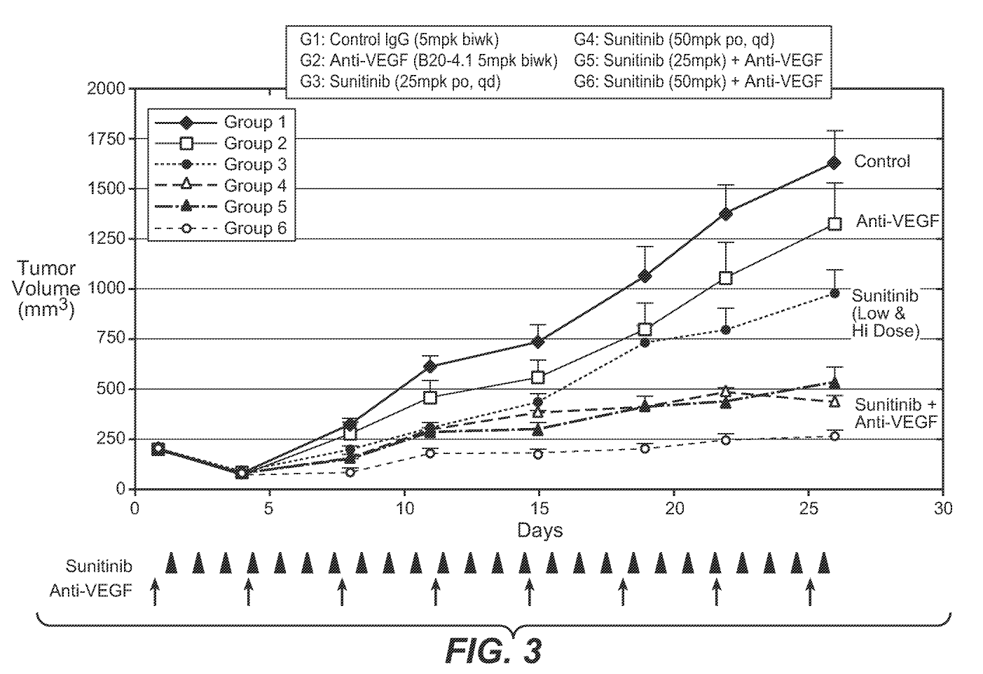

FIG. 3 represents the result from a longer term treatment study of the H1299 NSCLC tumors. The study also compared effects between two sunitinib doses—a low dose at 25 mg/kg daily and a high dose at 50 mg/kg daily. TGI was calculated using the measurement data from Day 19, when all mice still remained in the study. B20-4.1 monotherapy produced 64% TGI, corresponding to therapeutic activity, with no regression responses. Treatment with 25 or 50 mg/kg sunitinib produced dose-dependent TGIs of 32 and 62%. The 50 mg/kg sunitinib treatment group had one partial regression. Treatment with the combination of B20-4.1 and 25 or 50 mg/kg sunitinib produced TGIs of 74 and 82%, respectively with two partial regressions in each group. Thus, the combination treatment at either the low sunitinib dose or the high sunitinib dose provided significantly improved inhibition activity than the corresponding B20-4.1 and sunitinib single agent treatments. Especially significant is that at the low dose, sunitinib as a single agent failed to exert therapeutic activity in this study, whereas the B20-4.1/sunitinib combination at the same sunitinib dose provided significant inhibition to the NSCLC tumor growth. All treatments appeared to be well-tolerated, with no evidence of toxicity based upon body weight measurements or clinical symptoms.

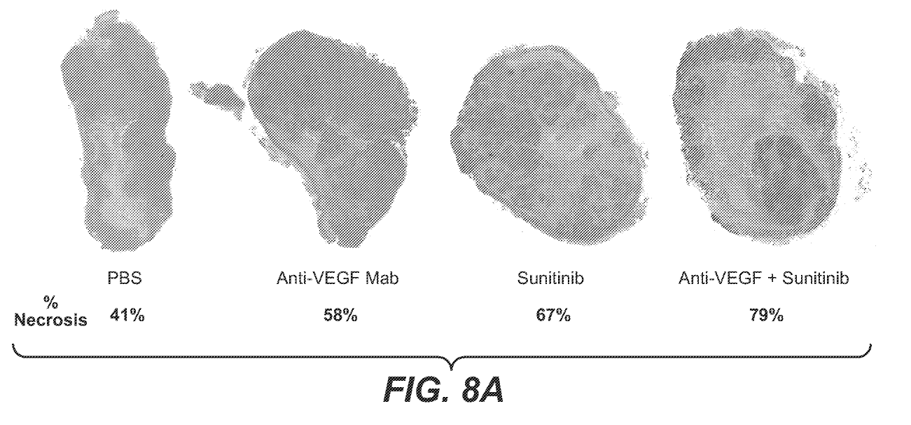

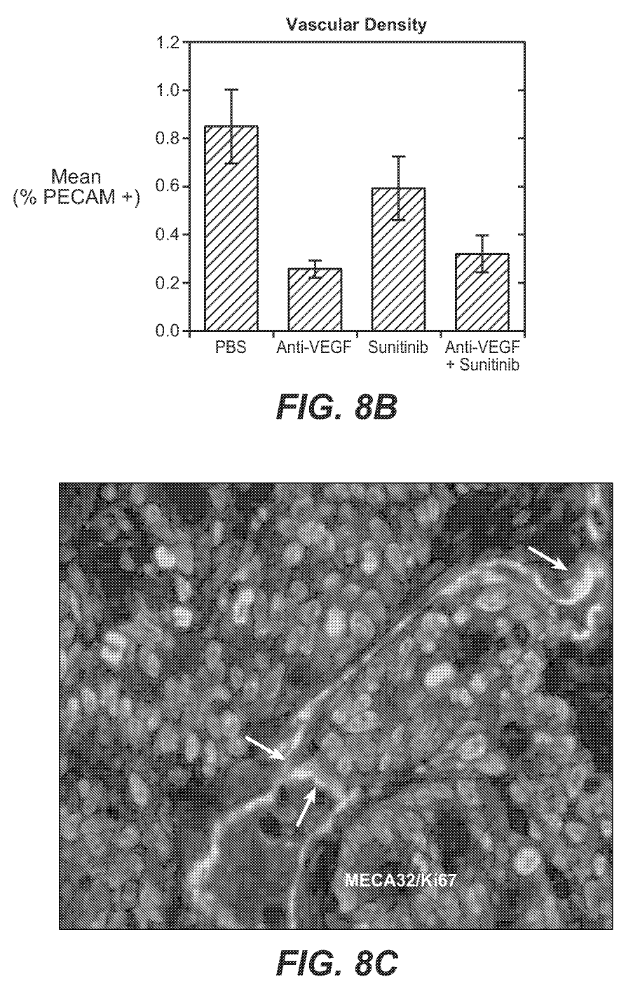

In addition to the TGI measurements, tumor samples were used in histological assays to compare the morphlogical changes of tumors under monotherapies vs. combination. Tumor necrosis and vascular density were measured at the end of the study. As shown in FIGS. 8A-8C, the B20-4.1/sunitinib combination results in markedly increased tumor necrosis and decreased microvascular density.

### Inhibition of Renal Cell Carcinoma Growth

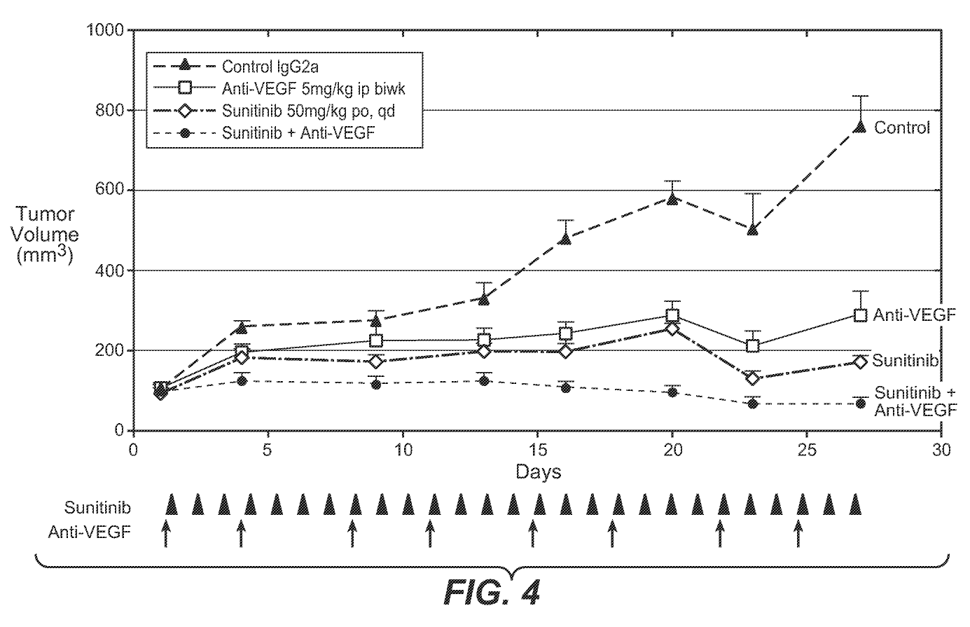

Two xenograft lines were used in the tumor growth study for human renal cell carcinoma. FIG. 4 shows the result from a study using the 786-O renal cell carcinoma xenografts. While both low and high doses of sunitinib were used in the study, only the high sunitinib dose (50 mg/kg daily) is shown in the figure for both the single agent group and the combination with B20-4.1. In this study, B20-4.1 monotherapy resulted in a 67% TGI, sunitinib monotherapy resulted in an 80% TGI and the combination showed a 92% TGI. The activity of the combination therapy was significantly better than single agent sunitinib or B20-4.1 (p=0.0002, p<0.0001, respectively). The combination also showed superior efficacy (TGI=77%) even when a lower dose of sunitinib (25 mg/kg qd) was combined with anti-VEGF therapy (p=0.0029).

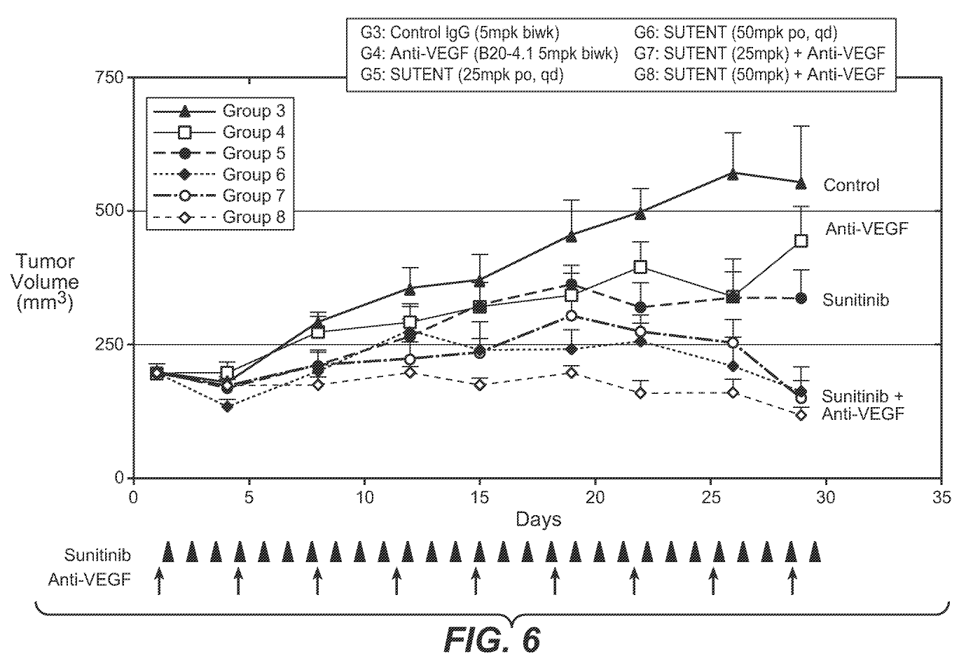

FIG. 6 shows the result from a study using the Caki-2 human renal cell carcinoma xenografts in SCID mice. Both low and high sunitinib doses are shown. TGI was calculated at Day 29, which was the last day of the study. Notably, both B20-4.1 monotherapy and the low dose (25 mg/kg) sunitinib monotherapy resulted in Day 29 mean tumor volumes (MTVs) of 446 and 352 mm³, which did not translate to therapeutic TGI under the study's definition. In comparison, the B20-4.1/sunitinib combination treatments produced a therapeutic TGI of 65%, which is a significant and meaningful improvement over either agent given alone (P<0.001). The high dose (50 mg/kg) sunitinib monotherapy did produced a 62% TGI, whereas the B20-4.1/sunitinib combination treatment at the same dose produced a increased TGI of 73%. No regression responses were documented in any group. The combination of B20-4.1 with 50 mg/kg 0-025694 was significantly better than B20-4.1 monotherapy, but not 50 mg/kg sunitinib monotherapy. However, the mean and median tumor growth curves suggested a trend toward combination activity. All treatments appeared to be well-tolerated, with no evidence of toxicity based upon body weight measurements or clinical symptoms.

### Inhibition of Pancreatic Carcinoma Growth

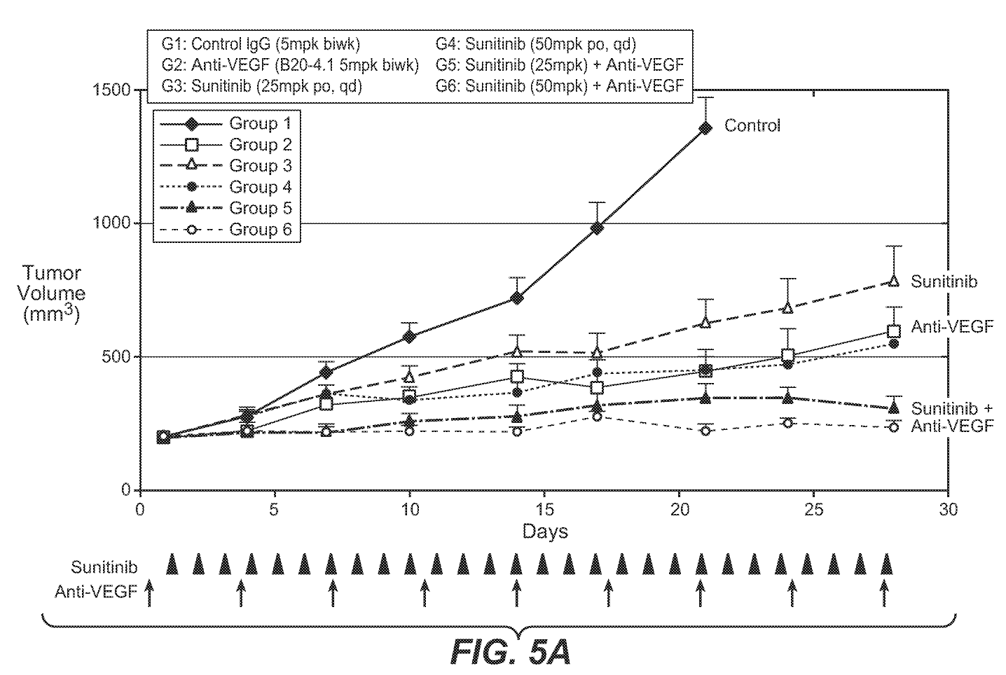

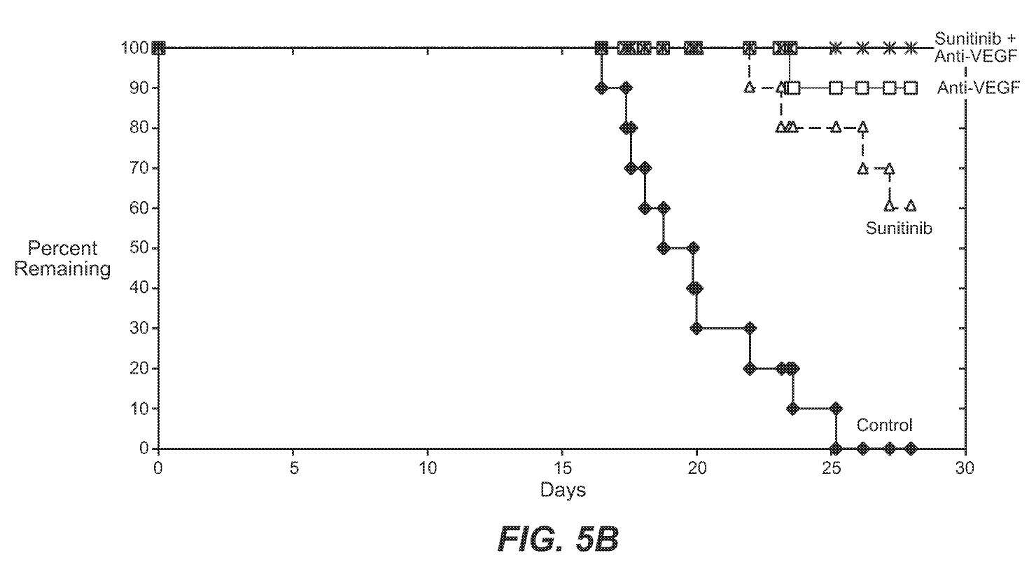

A study of tumor growth in Bx-PC3 human pancreatic carcinoma xenografts was carried out using agents and methods similar to the other studies as described above. FIGS. 5A and 5B show the result. Similar to the study in Caki-2 renal cell carcinoma xenografts, combination of B20-4.1 and sunitinib seems to produce more significant improvement over single agents when the low dose of sunitinib was administered. Compare Group 5 to either Group 3 or Group 2. While not wishing to be bound by theory, the results from this and the Caki-2 study may suggest that while at both doses sunitinib exhibit strong blocking activity of the PDGFR, it only exhibit effective blocking of the VEGFR activity at the high dose; so that the combination with B20-4.1 at the low dose provided more significant inhibition than the single agent. Such finding provides a strong rationale for combining anti-VEGF agent and sunitinib in treating tumors that respond poorly to the clinical dose of either monotherapy, or when a lower dose of sunitinib is more desirable.

FIGS. 7A and 7B further illustrate the combination effects when low dose sunitinib (25 mg/kg daily) was used in the Caki-2 RCC (FIG. 7A) and H1299 NSCLC (FIG. 7B) models.

Taken together, these xenograft mouse studies provide strong evidence for combination therapies wherein both VEGF and PDGF pathways are effectively targeted, resulting in additive or even synergistic antitumor activity and therefore significantly improved clinical outcome.

### Example 2

### Combination of Anti-VEGF and Sunitinib to Treat Breast Cancer

Breast cancer accounts for almost one-third of all new cancer diagnoses among women in the United States, with over 214,000 cases diagnosed in 2006. It is estimated that more than 41,000 women will die each year from their disease. Jernal et al., CA Cancer J. Clin 56:106-30 (2006). Despite many advances in the adjuvant treatment of early-stage diseases, up to 30-40% of women will develop systemic relapse. A small fraction of women with metastatic breast cancer (MTC) will be alive at 5 years, while only 2-3% become long-term survivors. Greenburg et al., J. Clin. Oncol. 14:2197-205 (1996).

This example provides a method of treating breast cancer with a combination of anti-VEGF, chemotherapy and sunitinib, which can result in prolong survival and improve quality of life, by administering to a subject an effective dose of bevacizumab, sunitinib and pacilitaxel. For example, in certain embodiments, a subject is administered: (1) bevacizumab at 10 mg/kg (e.g., based on subject's weight at Day 1) by IV infusion on day 1 and day 15 out of a 28 day cycle, (2) pacilitaxel at a dose of 65 mg/m²-90 mg/m² (typically, 90 mg/m²) by IV fusion every week for 3 weeks followed by one week rest, and (3) sunitinib, typically administered orally, at a dose of 20-50 mg/day, e.g., 25 mg/day or 37.5 mg/day, daily for 3 weeks followed by one-week rest period. In certain embodiments, a treatment cycle is defined as 4 weeks. Alternatively, sunitinib can be administered to the subject at a dose of 50 mg/day given according to a 4 weeks on, 2 weeks off schedule. Farve et al., J. Clin. Oncol. 24(1):25-35 (2006). Typically, bevacizumab comes in a 400-mg glass vial, which contains 16 ml of bevacizumab (25 mg/ml) with a vehicle consisting of sodium phosphate, trehalose, polysorbate 20 and sterile water for injection. Paclitaxel is available as a concentrated solution of 6 mg/ml in polyoxyethylated castor oil (Cremophor EL) 50% and dehydrated alcohol 50% in 5-, 16.7-, and 50-ml vials. Sutent® is the malate salt of sunitinib. Sunitinib malate is described chemically as butanedioic acid, hydroxy-(2S)—, compounded with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1). The molecule formula is C₂₂H₂₇FN₄O₂—C₄H₆O₅. Sunitinib capsules contain sunitinib malate equivalent to 12.5 mg or 25 mg of sunitinib with mannitol, croscarmellose sodium, povidone (K-25), and magnesium stearate as inactive ingredients.

An outcome measure is progression free survival (PFS) based on the analysis (e.g., done by Independent Review Facility (IRF) assessment) of tumor response, which can be assessed by Response Evaluation Criteria in Solid Tumors (RECIST) and/or IRF (radiographs). Therasse et al., J. Natl Cancer Inst. 92:205-16 (2000). Overall survival, 12-month survival, objective response, duration of objective response and 12 month PFS can also be used as an outcome measure. Plasma levels of soluble proteins can also be evaluated, e.g., sVEGFR2, s VEGFR3 and VEGF-C and the PDGF pathway.

Subjects for the methods of treatment are subjects that have adenocarcinoma of the breast (e.g., determined by histological or cytological studies). Typically, the subjects have measurable or non-measurable locally recurrent or metatastic disease. In certain embodiments, the locally recurrent disease should not be amenable to resection with curative intent. In certain embodiments, the subjects may have received prior hormonal therapy, e.g., in either the adjuvant or metastatic setting (e.g., if discontinued ≧2 weeks prior to Day 1). In certain embodiments, the subject may have received adjuvant non-taxane chemotherapy (e.g., if discontinued ≧6 months prior to starting the program). In certain embodiments, the subject may have received adjuvant taxane chemotherapy (e.g., if discontinued ≧12 months prior to Day 1). Optionally, subjects may also receive concurrent bisphosphonate therapy (e.g., if started prior to or within the first 30 days of study entry). Optionally, subject may also have received prior radiotherapy when starting the treatment, provided the subject has recovered from any significant (Grade ≧to 3) acute toxicity prior to Day 1.

In certain embodiments, excluded subjects are subject with unknown HER2 or known HER2-positive status. In general, HER2-positive status can be identified by a fluorescence in situ hybridization (FISH) assay, or by a 3+immunohistochemistry result by a method known in the art. Other excluded subjects include subjects with prior chemotherapy for locally recurrent or metastatic disease, subjects with prior hormonal therapy within 2 weeks prior to Day 1, subjects with prior adjuvant or neoadjuvant taxane chemotherapy within 12 months prior to Day 1, subject with prior adjuvant or neoadjuvant non-taxane chemotherapy within 6 months prior to Day 1, or subjects who had received recent radiotherapy, ongoing Grade ≧3 acute toxicity. Excluded subjects also include subjects with inadequate organ function (e.g., evidenced by reduced neutrophil count, a reduced platelet count, or a total bilirubin of greater than 1.5 mg/dl), with uncontrolled serious medical or psychiatric illness, with inadequately controlled hypertension (e.g., defined as systolic blood pressure greater than 150 mmHg and/or diastolic blood pressure greater than 100 mmHg on anti-hypertensive medications), with prior history of hypertensive crisis or hypertensive encephalopathy, with a history of myocardial infarction or unstable angina within 12 months prior to Day 1, with a history of stroke or transient ischemic attack within 12 months prior to Day 1, with evidence of bleeding diathesis or significant coagulopathy, or with serious, non-healing wound, active ulcer, or untreated bone fracture. See also additional exclusions on labels of bevacizumab, and sunitinib.

### Example 3

### Combination of Anti-VEGF and Sunitinib to Treat Advanced Non-Small Cell Lung Cancer

Lung cancer is the leading cause of cancer death in the United States, with an incidence of 174,470 new cases and 162,460 deaths estimated to occur in 2006. Approximately 55%-75% of patients with non-small cell lung cancer (NSCLC) present with advanced disease (unresectable or metastatic disease). The overall 5-year survival rate for patients with lung cancer in the United States is 14%, having only slightly improved over the past 20 years. DeVita et al., Cancer: principles and practice of oncology, 6^{th} ed. Philadelphia (Pa.): Lippincott Williams and Wilkins; 2001. Patients who present with Stage IIIb and Stage IV disease have 5-year survival rates of 6% and 8%, respectively. Mountain, Chest 111: 1710-7 (1997). After definitive initial treatment, which consists of surgical resection, radiotherapy, chemotherapy, or combinations of these modalities, approximately 50% of patients with early-stage disease and 80% of those with locally advanced disease will relapse and present for first-line or later-line treatment.

This example provides a method of treating non-small cell lung cancer with a combination of anti-VEGF, chemotherapy and sunitinib, which can result in prolong survival and improve quality of life, by administering to a subject an effective dose of bevacizumab, sunitinib, pacilitaxel and carboplatin. For example, in certain embodiments, a subject is administered: (1) bevacizumab at 15 mg/kg (e.g., based on subject's weight at Day 1) by IV infusion on day 1 of a 21 day cycle, (2) carboplatin as dosed by area under the curve (AUC=6) based on the Calvert formula (discussed in detail below), (3) pacilitaxel at a dose of 200 mg/m² (e.g., based on subject's weight at Day 1) by IV fusion on day 1 of each 21-day cycle for a total of four cycles, and (4) sunitinib, typically administered orally, at a dose of 20-50 mg/day, e.g., 25 mg/day or 37.5 mg/day, daily for 2 weeks followed by one-week rest period. In certain embodiments, a treatment cycle is defined as 3 weeks. Alternatively, sunitinib can be administered to the subject at a dose of 50 mg/day given according to a 4 weeks on, 2 weeks off schedule.

As discussed above, carboplatin is dosed by area under the curve (AUC=6) based on the Calvert formula:

Total Dose(mg)=(target AUC)×(GFR+25),

where GFR is the glomerular filtration rate in milliliters per minute (mL/min) and the target area under the curve (AUC) is 6 mg/mL×min. GFR as creatinine clearance may be measured (preferably) via a 24-hour urine collection or may be calculated based on serum creatinine using the Cockcroft-Gault formula (Cockcroft and Gault 1976). For males, GFR is estimated as follows:

GFR=(140−age)×weight/[72×(serum creatinine)].

For females, GFR=0.85 times this formula. Age is in years, weight is in kilograms, and serum creatinine is in milligrams per deciliter.

Typically, bevacizumab comes in a 400-mg glass vial, which contains 16 ml of bevacizumab (25 mg/ml) with a vehicle consisting of sodium phosphate, trehalose, polysorbate 20 and sterile water for injection.

Carboplatin is available as a premixed sterile aqueous solution of 10 mg/mL ready for dilution and parenteral administration. Vials are available in 50-, 150-, 450-, and 600-mg sizes.

Paclitaxel is available as a concentrated solution of 6 mg/mL in polyoxyethylated castor oil (Cremophor EL) 50% and dehydrated alcohol 50% in 5-, 16.7-, and 50-mL vials.

Sunitinib malate is described chemically as butanedioic acid, hydroxy-(2S)—, compounded with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1). The molecular formula is C₂₂H₂₇FN₄O₂.C₄H₆O₅. Sunitinib capsules contain sunitinib malate equivalent to 12.5 mg or 25 mg of sunitinib with mannitol, croscarmellose sodium, povidone (K-25), and magnesium stearate as inactive ingredients.

An outcome measure is progression free survival (PFS) based on the analysis (e.g., done by Independent Review Facility (IRF) assessment) of tumor response, which can be assessed by Response Evaluation Criteria in Solid Tumors (RECIST) and/or IRF (radiographs). Overall survival, objective response, duration of objective response can also be used as an outcome measure. During the course of the treatment, plasma samples can be drawn from the patients for measuring plasma levels of prognostic and predictive biomarkers such as the molecules implicated in the angiogenesis pathway, including but not limited to VEGF, soluble VEGFRs, PDGF and soluble PDGFRs.

Subjects for the methods of treatment are subjects that have locally advanced, recurrent, or metastatic squamous NSCLC (e.g., determined by histological or cytological studies). In one embodiment, excluded subjects are subjects with prior systemic chemotherapy for metastatic disease. Other excluded subjects include subjects with active malignancy other than lung cancer, subjects with current, recent (within 4 weeks of Day 1), or planned participation in another experimental drug study, and subjects with prior treatment with anti-VEGF agent or agents targeting similar pathways as sunitinib. Other exclusion criteria may also be used, including general medical exclusions or those typical for bevacizumab and sunitinib therapies.

### Example 4

### Combination of Anti-VEGF and Sunitinib to Treat Renal Cell Cancer

Renal cell cancer (RCC) constitutes approximately 2% of all malignancies, with an estimated incidence of 39,000 cases per year and approximately 13,000 deaths per year in the United States. Until recently, the effectiveness of treatments for metastatic disease has been dismal. Such treatments included cytokines (interferon α [IFNα] and interleukin-2 [IL-2]), which provided little benefit and a great degree of toxicity. The latter was the only approved agent for this disease until late 2005. New treatments have emerged for patients with metastatic RCC with two recently approved agents, sunitinib (Sutent®) and sorafenib (Nexavar®).

This example provides a method of treating renal cell cancer with a combination of anti-VEGF and sunitinib, which can result in prolonged survival and improved quality of life, by administering to a subject an effective dose of bevacizumab and sunitinib.

For example, in certain embodiments, a subject is administered: (1) bevacizumab at 10 mg/kg (e.g., based on subject's weight at Day 1) by IV infusion every 2 weeks on day 1, 15 and 29 of each 42 day (6 week) cycle, and (2) sunitinib, typically administered orally, at a dose of 50 mg/day for 4 weeks followed by a 2-week rest period. In certain embodiments, a treatment cycle is defined as 4 weeks of sunitinib and 2 weeks of rest (6 weeks).

Typically, bevacizumab comes in a 400-mg glass vial, which contains 16 ml of bevacizumab (25 mg/ml) with a vehicle consisting of sodium phosphate, trehalose, polysorbate 20 and sterile water for injection.

Sunitinib malate is described chemically as butanedioic acid, hydroxy-(2S)—, compounded with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1). The molecular formula is C₂₂H₂₇FN₄O₂.C₄H₆O₅. Sunitinib capsules contain sunitinib malate equivalent to 12.5 mg or 25 mg of sunitinib with mannitol, croscarmellose sodium, povidone (K-25), and magnesium stearate as inactive ingredients.

An outcome measure is progression free survival (PFS) based on the analysis (e.g., done by Independent Review Facility (IRF) assessment) of tumor response, which can be assessed by Response Evaluation Criteria in Solid Tumors (RECIST) (Therasse et al., New guidelines to evaluate the response to treatment in solid tumors, J. Natl Cancer Inst. 2000:92:205-16, and/or IRF (radiographs). Overall survival, objective response and duration of objective response can also be used as an outcome measure. During the course of the treatment, plasma samples can be drawn from the patients for measuring plasma levels of prognostic and predictive biomarkers such as the molecules implicated in the angiogenesis pathway, including but not limited to VEGF, soluble VEGFRs, PDGF and soluble PDGFRs.

In one embodiment, subjects for the methods of treatment are subjects that have histologically confirmed metastatic RCC that is predominantly clear cell (>50%). In another embodiment, the subjects include subjects with measurable (lesions that can be accurately measured in at least one dimension (longest diameter to be recorded) as 20 mm with conventional techniques or as 10 mm with spiral CT scan) disease as defined by RECIST. In yet another embodiment, the subjects include subjects with prior nephrectomy.

In one embodiment, excluded subjects are subjects with RCC with predominantly sarcomatoid features. Other excluded subjects include subjects with prior systemic or adjuvant therapy for RCC. In yet another embodiment, excluded subjects are subjects underwent radiotherapy for RCC within 2 days prior to Day 1, with the exception of single-fraction radiotherapy given for the indication of pain control. In yet another embodiment, excluded subjects may include those with current need for dialysis and those who are already undergoing treatment with bevacizumab, sunitinib, sorafenib, axitinib, thalidomide, or other agents, either investigational or marketed, that act by either VEGF inhibition or anti-angiogenesis mechanisms. Other exclusion criteria may also be used, including general medical exclusions or those typical for bevacizumab and sunitinib therapies.

What is claimed is:

1. A method of treating tumor in a subject, comprising administering to said subject a VEGF antagonist and a protein kinase inhibitor,
wherein said VEGF antagonist interferes the binding of VEGF to a cellular receptor,
wherein said protein kinase inhibitor is capable of inhibiting at least the PDGF receptor tyrosine kinase, and
wherein said administering is for a time and in an amount sufficient to treat or prevent said tumor in said subject.

2. The method of claim 1, wherein said VEGF antagonist is an aptamer capable of specifically binding to VEGF.

3. The method of claim 1, wherein said VEGF antagonist is a soluble VEGF receptor protein, or VEGF binding fragment thereof, or a chimeric VEGF receptor protein.

4. The method of claim 3, wherein said chimeric VEGF receptor protein comprises at least the extracellular domain 2 from Flt-1 or KDR.

5. The method of claim 1, wherein said VEGF antagonist is an anti-VEGF antibody.

6. The method of claim 5, wherein said anti-VEGF antibody is a monoclonal antibody.

7. The method of claim 6, wherein said monoclonal antibody is a chimeric, fully human, or humanized antibody.

8. The method of claim 7, wherein said anti-VEGF antibody is bevacizumab, G6 series antibody, B20 series antibody, or VEGF binding fragments thereof.

9. The method of claim 8, wherein said anti-VEGF antibody is bevacizumab.

10. The method of claim 1, wherein said protein kinase inhibitor is capable of inhibiting both PDGF receptor tyrosine kinase and VEGF receptor tyrosine kinase.

11. The method of claim 10, wherein said protein kinase inhibitor is sunitinib or a pharmaceutically acceptable salt form thereof.

12. The method of claim 1, wherein the tumor is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma.

13. The method of claim 12, wherein the tumor is non-small cell lung cancer.

14. The method of claim 12, wherein the cancer is metastatic.

15. The method of claim 12, wherein the patient is previously untreated.

16. The method of claim 1, wherein the VEGF antagonist is bevacizumab and the protein kinase inhibitor is sunitinib.

17. The method of claim 16, wherein bevacizumab is administered intravenously to the subject at 10 mg/kg every other week or 15 mg/kg every three weeks, and wherein sunitinib is administered orally to the subject at a daily dose of 25 mg for 2, 3 or 4 weeks followed by 1 or 2 weeks off.

18. The method of claim 17, wherein bevacizumab is administered intravenously to the subject at 15 mg/kg every three weeks, and wherein sunitinib is administered orally to the subject at a daily dose of 25 mg for 2 weeks followed by 1 week off.

19. The method of claim 1 or 16, further comprising administering to the subject one or more chemotherapeutic agent(s).

20. The method of claim 19 wherein the chemotherapeutic agent administered is paclitaxel.

21. The method of claim 19 wherein the chemotherapeutic agents administered are carboplatin and paclitaxel.
